(19) United States
(12) Patent Application Publication
(10) Pub. No.: US 2006/0282121 A1
(43) Pub. Date: Dec. 14, 2006

(54) Vagus nerve stimulation for chronic intractable hiccups

(76) Inventors: Bryan R. Payne, New Orleans, LA (US); Robert L. Tiel, New Orleans, LA (US)

Correspondence Address: WARNER J DELAUNE JR;ADAMS AND REESE LLP, 450 LAUREL STREET, SUITE 1900, BATON ROUGE, LA, 70801, US

(21) Appl. No.: 11/410,860

(22) Filed: Apr. 24, 2006

Related U.S. Application Data
(60) Provisional application No. 60/674,519, filed on Apr. 25, 2005.

(51) Int. Cl. A61N 1/18

(52) U.S. Cl. 607/2; 607/42

(57) ABSTRACT

Intractable hiccups are debilitating and are usually a result of underlying disease. A method of treating chronic hiccups is provided, comprising the steps of applying a peripheral nerve stimulator to the vagus nerve at a predetermined frequency, pulse width, amplitude, and duration; monitoring the occurrence of hiccups in response to said stimulation; and adjusting said frequency, pulse width, amplitude, and duration to reduce the occurrence of said hiccups. The vagus nerve stimulation methods disclosed herein have provided complete resolution to date with a patient with intractable hiccups occurring after a posterior fossa stroke.

### RELATION TO PRIOR PATENT APPLICATION

This nonprovisional patent application claims the benefit under 35 U.S.C. §119(e) of provisional patent application Ser. No. 60/674,519, filed on Apr. 25, 2005.

### BACKGROUND OF THE INVENTION

I. Field of the Invention

This invention relates to the use of vagus nerve stimulation to alleviate chronic intractable hiccups.

II. Prior Art

The hiccup is a universal experience, but is in fact an incompletely understood neurophysiologic phenomenon. It is easily described as a sudden powerful activation of the inspiratory muscles of the thorax, diaphragm, neck accessory, and external intercostals muscles, with brief inhibition of the expiratory muscles, active movement of the tongue toward the roof of the mouth and active adduction of the vocal cords, which begins after the initiation of inspiratory flow. The English name “hiccup” as well as the French “hocquet” is onomatopoeic for the sound which results from this forced inspiration against a closed glottis. When hiccups become intractable (singultus) this otherwise benign curiosity can lead to insomnia, wasting, exhaustion and even death.

The hiccup reflex arc is customarily divided into an afferent limb, a central connection, and an efferent limb. The afferent pathways are comprised of vagal, phrenic and sympathetic (T6-12) branches. The efferent pathways are comprised of the phrenic nerve to the diaphragm, direct plexal branches to the scalene muscles, vagal branches to the glottis, and intercostals nerves to the external intercostals muscles. The central connection is then the spinal cord from the C3-5 spinal levels rostral to the medulla oblongata. As a consequence of the coordinated activation of all the inspiratory muscles with a hiccup and the maintenance of breathing while hiccupping, it has been speculated that there is a rhythm center responsible for the generating of hiccups that is separate from the adult mammalian respiratory generator, but which may interact with it.

The automatic, phasic and continuous contraction of the respiratory muscles results from the output of neural networks located in the brainstem. There is evidence to support the localization of a pacemaker center in the Pre-Botzinger complex, just caudal to the retrofacial nucleus and ventral to the nucleus ambiguous. Other work suggests that the respiratory rhythm results for emergent properties of neuronal networks. In the cat, electrical stimulation between the nucleus ambiguous and the lateral reticular nucleus has been demonstrated to induce hiccups. The presence of intra-utero hiccups has fueled speculation that there may be a phylogenitical explanation of this reflex as a vestige of an archaic gill ventilation pattern seen in frogs of the tadpole stage. The fact that this form of gill-ventilation can be arrested by baclofen, which is used frequently in the medical management of hiccups without affecting respiration pathways, lends further credence to this interesting hypothesis.

Pathologic or physiologic dysfunction of either the afferent or efferent neural limbs has been more often found to be an initiator of episodic or intractable hiccups. Brainstem pathology while distinctly less common has been clearly associated with central neurogenic causes for hiccups. We describe a case report of a patient with intractable hiccups central in origin successfully treated with vagus nerve stimulation.

### DETAILED DESCRIPTION OF THE PREFERRED EMBODIMENTS

The case study relates to a 51-year-old male who presented with a 7-month history of intractable hiccups. Two years prior to his initial presentation in a clinic, he suffered three successive cerebellar strokes requiring decompressive craniectomy. No etiology of the strokes was discovered and the patient was prophylactically placed on anticoagulation therapy utilizing Plavix (clopidogrel bisulfate). Immediately following his stroke hiccups began occurring for periods up to one month. Most recently his hiccups occurred for seven months. Hiccups occurred approximately every 4-5 seconds while awake and during sleep. Multiple home remedies, as well as recommended medical therapies such as chlorpromazine, metoclopramide, domperidone, papaverine, and baclofen, were unsuccessful. Trials of other medications including cyclobenzaprine (Flexeril) and tizanidine (Zanaflex) provided no relief. His family practitioner prescribed butorphanol tartrate (stadol) for an unrelated episode of left leg pain, which coincidentally relieved hiccups for about 30 minutes. The patient thereafter self-administered intramuscular stadol injections up to 12 times daily in order to experience short episodes of relief. Self-induced vomiting also produced short periods without hiccups lasting approximately 30 minutes.

After 7 months of chronic intractable hiccups, the patient presented to our neurosurgical clinic for further evaluation and treatment. Initial studies included an MRI of the brain, which revealed a large zone of encephalomalacia of the left cerebellar hemisphere extending to the vermis inferiorly with some sparing of the superior medial left cerebellar hemisphere. Blood chemistries were unremarkable. Fluoroscopy of the chest revealed bilateral diaphragm involvement concurrent with hiccupping. A series of peripheral nerve blocks were then done using fluoroscopic imaging. A right phrenic nerve block with 10 cc of 25% mancurin paralyzed the right hemidiaphragm, however, hiccups remained present. Ten minutes after the nerve block, hiccups were relieved as the patient's voice became hoarse. These findings suggest a right recurrent laryngeal nerve block associated with a right vagus nerve block, achieved by local extension of the paralytic agent. Restoration of nerve function 6 hours later corresponded with a return of right hemidiaphragm function, normal voice, and hiccups. The next day left phrenic nerve percutaneous pacing was performed with no change in diaphragm movement or hiccup occurrence. Subsequently, a left vagus nerve block was performed, which resulted in successful treatment of hiccups (associated with a hoarse voice) and lasted 45 minutes.

Based on the successes of the vagus nerve blocks, the decision was made to implant a Medtronics® peripheral nerve stimulator lead to the left vagus nerve, externalized for trial stimulation. The patient did not hiccup following surgery, except for 10 minutes in the immediate post-operative evening. His voice was noted to be hoarse while hiccups remained absent. No trial of stimulation was performed. Three days later, the patient returned to the operating room for removal of the peripheral nerve stimulator leads and placement of Cyberonics® vagus nerve stimulation (VNS) leads to the left vagus nerve. The generator powering the VNS leads was not placed at that time. Because the peripheral nerve stimulator lead was effective in treating hiccups merely with direct contact with the nerve and without stimulation, the physicians and patient agreed to placement of VNS leads without stimulation. The generator could be placed at a later date if hiccups were to return. However, the hiccups returned within four hours of surgery and the generator was implanted the following day. Hiccups returned several hours after this procedure. The following morning stimulation was begun with the following initial settings: 15 hertz, pulse width 750 microseconds, amplitude 1.5 milliamps, on 30 seconds, off 5 minutes. Complete relief of hiccups has been achieved to date.

VNS therapy has been used widely for treating patients with seizures, whose symptoms are not optimally controlled with antiepileptic medications. We present the first case of VNS therapy used in hiccup treatment.

Pharmacotherapy for intractable hiccups includes dopaminergic antagonists such as chlorpromazine and haloperidol, antiarrythmics such as nifedipine, lidocaine and phenytoin, and other medicines such as metoclopramide, baclofen, and gabapentin. Effective surgical management includes phrenic nerve blockade or pacing.

Although the phrenic nerve is believed to be the only motor nerve innervating the diaphragm, an accessory phrenic nerve may supplement it. In these cases, blockade of the phrenic nerve will not produce paralysis of the diaphragm. Furthermore, hiccups of central origin are associated with bilateral diaphragm contraction. A unilateral phrenic nerve block in these cases, while paralyzing the ipsilateral diaphragm, is unlikely to relieve hiccups. Bilateral phrenic nerve blockade may also be ineffective for hiccups of central origin. As mentioned previously, efferent pathways involved in hiccup movement are not only the phrenic nerves but also include direct plexal branches to the scalene muscles and intercostal nerves to the external intercostal muscles. Bilateral phrenic nerve blockade is also cautioned against because of the risk of pulmonary function compromise. Percutaneous phrenic nerve stimulation has also been reported in treating hiccups.

In our patient, administering a phrenic nerve block was effective only when a vagus nerve block was also achieved. The reasons for our success of vagus nerve blockade followed by stimulation in treating hiccups is not yet completely understood. The vagus nerve is involved in the afferent limb of the hiccup pathway and originates in the medulla. Therefore, signals are likely received from the vagus nerve through the medulla to the central hiccup center. VNS may alter signals transmitted through the medulla and into the hiccup rhythm center, thus ceasing hiccups. Possibly VNS was effective in our patient because of vagal nerve response and behavior in the presence of cerebellar disease. Two cases of cerebellar disease leading to hiccups have been reported. Hiccups were not relieved in either case.

Why the phrenic nerve blockade and stimulation were not beneficial to our patient is unknown. Reasons mentioned above for phrenic nerve blockade failure might apply in our case. Alternatively, perhaps the phrenic nerve, although also involved in the afferent limb of the hiccup pathway, does not transmit signals to the hiccup center as the vagus nerve does. Therefore, further clinical evaluation is needed to determine if vagal nerve intervention relieves hiccups because of its unique pathway to the hiccup center or because of our patient's underlying cerebellar pathology.

Although exemplary embodiments of the present invention have been shown and described; many changes, modifications, and substitutions may be made by one having ordinary skill in the art without necessarily departing from the spirit and scope of the invention.

We claim:

1. A method of treating chronic hiccups, comprising the steps of:
(a) applying a peripheral nerve stimulator to the vagus nerve at a predetermined frequency, pulse width, amplitude, and duration;
(b) monitoring the occurrence of hiccups in response to said stimulation; and
(c) adjusting said frequency, pulse width, amplitude, and duration to reduce the occurrence of said hiccups.

2. The method of claim 1, wherein said frequency is between 10 and 20 hertz.

3. The method of claim 1, wherein said pulse width is between 500 microseconds and 1000 microseconds.

4. The method of claim 1, wherein said amplitude is between 1.0 and 2.0 milliamps.

5. The method of claim 1, wherein said duration is between 1 and 60 seconds.
